(19) United States
(12) Patent Application Publication
(10) Pub. No.: US 2023/0215542 A1
(43) Pub. Date: Jul. 6, 2023

(54) PSYCHOLOGY COUNSELING DEVICE AND METHOD THEREOF

(71) Applicant: Haii Co, Ltd., Seoul (KR)

(72) Inventors: Hunyeop Jeong, Hanam-si, Gyeonggi-do (KR); Jeehang Lee, Goyang-si, Gyeonggi-do (KR); Jeonghwan Ahn, Seoul (KR); Sangho Jin, Incheon (KR); Chanmi Park, Seoul (KR); Gugji Yang, Goyang-si Gyeonggi-do (KR); Soohyun Moon, Seoul (KR)

(21) Appl. No.: 18/009,638

(22) PCT Filed: Apr. 22, 2022
(86) PCT No.: PCT/KR22/05801

§ 371 Date: Dec. 9, 2022

(30) Foreign Application Priority Data
Nov. 16, 2021 (KR) 10-2021-0158016

(51) Int. Cl. G16H 20/70; G16H 40/67; G16H 10/20; G06F 3/16

(52) U.S. Cl. CPC G16H 20/70; G16H 40/67; G16H 10/20; G06F 3/167

(57) ABSTRACT

A psychology counseling device is provided. The device includes a user interface configured to receive an input from a user and provide information; a microphone configured to collect a voice of the user; a speaker configured to convey auditory information to the user; a processor configured to control the user interface, the microphone, and the speaker; and a memory accessible by the processor and configured to store executable instructions. The memory is configured to further store texts to be provided to the user and voice data received from the user. The executable instructions, when executed by the processor, causes the processor to perform: recognizing an emotional state of the user based on the user's input; providing texts including different contents to the user according to the emotional state of the user; receiving a voice that the user articulates the texts and storing the voice in the memory as the voice data; obtaining a plurality of modulated voices by converting the voice data; and providing at least two among the plurality of modulated voices to the user.

### FIELD OF THE INVENTION

This application is a priority claim application for Korean Patent Application No. 10-2021-0158016 filed on Nov. 16, 2021, and all contents disclosed in the specification and the accompanying drawings thereof are incorporated herein by reference.

The present disclosure relates to a device and a method for alleviating a person's unstable psychological state, and more particularly, to a device and a method for enhancing user's psychological flexibility in a self-talk method by providing suitable contents for each user.

### BACKGROUND OF THE INVENTION

According to data released by the World Health Organization in 2018, more than 300 million people worldwide each suffer from depression and anxiety. In particular, the number of people experiencing depression and anxiety is increasing rapidly due to the recent pandemic situation caused by the coronavirus disease (COVID)-19 virus. As depression or anxiety progresses, it can affect an individual's physical functioning and performance. The need to alleviate depression and anxiety which limit these mental and physical activities is highly demanded. However, it is difficult for people suffering from depression and anxiety to actively participate in treatment due to various reasons such as people's negative views, fear of social stigma, and financial burden.

Recently, many psychological counseling services and mindfulness have been introduced through smartphone applications which are highly accessible and have a low financial burden for psychological solutions, and user's need and interest in the above services are growing. However, in the case of psychological counseling through an application, there are limitations in that it is difficult to provide real-time and there is a concern in that the user reveals his/her information to others even when the psychological counseling is performed in a non-face-to-face manner.

In addition, in the case of wellness services such as mindfulness, there are provided many distraction techniques for providing contents to users, specifically, people who suffer from depression and anxiety, to shift their attention to external information that is not related to themselves so that they can break free from maladaptive self-focus. However, the method of distracting attention to the external information requires too much difficulty for people who suffer from psychological difficulties such as depression and anxiety, and by increasing the repression and avoidance of thinking about themselves, depression and anxiety may recur. Thus, the method is pointed out that there is a limit to a long-term and sustainable solution.

That is, currently, a method of unidirectional consumption of wellness services such as non-face-to-face counseling and mindfulness as a method of alleviating depression and anxiety through applications is limited and does not provide a highly effective solution.

### RELATED ART DOCUMENTS

### Patent Documents

(Patent Document 1) Korean Patent Registration No. 10-1683310
(Patent Document 2) Korean Patent Registration No. 10-1689021
(Patent Document 3) Korean Patent Registration No. 10-1706123
(Patent Document 4) Korean Patent Publication No. 10-2020-0065248
(Patent Document 5) Korean Patent Publication No. 10-2018-0060060
(Patent Document 6) Korean Patent Publication No. 10-2019-0125154
(Patent Document 7) Korean Patent Publication No. 10-2020-0113775

### SUMMARY OF THE INVENTION

The present disclosure is for the purpose of providing a psychology counseling device that a user experiencing symptoms of depression or anxiety is capable of monitoring emotional information by itself and a method therefor. In addition, another purpose of the present disclosure is to provide a device and a method for providing customized contents to each user experiencing symptoms of depression or anxiety and providing psychological counseling through self-talk by listening to a user's own voice.

According to an aspect of the present disclosure, there is provided a psychology counseling device which including: a user interface configured to receive input from a user and provide information; a microphone for collecting a voice of the user; a speaker configured to convey auditory information to the user; a processor for controlling the user interface, the microphone, and the speaker; and a memory accessible by the processor and configured to store executable instructions. The memory is configured to further store texts to be provided to the user and voice data received from the user. The executable instructions, when executed by the processor, causes the processor to perform operations of: recognizing an emotional state of the user based on the user's input; providing texts including different contents to the user according to the emotional state of the user; receiving the voice that the user articulates the texts and storing the voice in the memory as voice data; obtaining a plurality of modulated voices by converting the voice data; and providing at least two of the plurality of modulated voices to the user. wherein the memory is configured to further store texts to be provided to the user and voice data received from the user;

In one embodiment of the present disclosure, the recognizing of an emotional state of the user based on the user's input may include: providing icons or words indicating emotional states to the user; and receiving an icon or word selected by the user.

In one embodiment of the present disclosure, the icon or word indicating of the emotional state may indicate positive, negative or neutral; and wherein the providing texts including different contents to the user according to the emotional state of the user may include one of: in response to the user's emotional state being positive, providing a text based on Positive Self-Talk (PST); in response to the user's emotional state being negative, providing a text based on a Cognitive Behavioral Therapy (CBT) method; and in response to the user's emotional state being neutral, providing a text based on mindfulness and breathing.

In one embodiment of the present disclosure, the recognizing of an emotional state of the user based on the user's input may include: providing a momentary assessment question to the user; receiving a response to the momentary assessment question from the user; and recognizing the emotional state based on the question and the response.

In one embodiment of the present disclosure, the obtaining of a plurality of modulated voices by converting the voice data may include: automatically performing according to a predetermined rule in response to the user receiving the voice that the user articulates the texts and storing the voice as voice data. Further, the automatically performing according to a predetermined rule may include: providing modulated voices to the user, wherein the modulated voices include a first type in which both a pitch and a formant of the voice data are increased, a second type in which the pitch of the voice data is decreased and the formant of the voice data is increased, a third type in which the pitch and the formant of the voice data are decreased, and a fourth type in which the pitch of the voice data is increased and the formant of the voice data is decreased.

In one embodiment of the present disclosure, the obtaining of a plurality of modulated voices by converting the voice data may include: providing modulated voices to the user, wherein the modulated voices include a first type in which both the pitch and the formant of the voice data are increased, a second type in which the pitch of the voice data is decreased and the formant of the voice data is increased, a third type in which the pitch and the formant of the voice data are decreased, and a fourth type in which the pitch of the voice data is increased and the formant of the voice data is decreased; receiving any one of the first to fourth types selected by the user; determining which element of the pitch and the formant the user wants to adjust based on the type selected by the user, including: providing keywords corresponding to the first to fourth types to the user; and in response to receiving the keyword selected by the user, adjusting the pitch and the formant of the voice data and providing it to the user.

In one embodiment of the present disclosure, the providing of at least two of the plurality of modulated voices to the user may include: further providing the user with the voice that the user articulates the texts.

In one embodiment of the present disclosure, the texts provided to the user may include any one of a text based on Positive Self-Talk (PST), a text based on a Cognitive Behavioral Therapy (CBT) method, and a text based on mindfulness and breathing.

According to another aspect of the present disclosure, there is provided a psychological counseling method using a psychological counseling device that provides self-talk to a user is provided, wherein the psychological counseling device includes a user interface, a memory, a microphone, a speaker, and a processor for controlling the user interface, the memory, the microphone, and the speaker. The method comprises: by the processor, recognizing an emotional state of the user based on the user's input through the user interface; reading texts including different contents according to the emotional state of the user from the memory and providing them to the user; by the microphone, receiving the user's voice uttering the text with the microphone and storing it as voice data in the memory; by the processor, obtaining a plurality of modulated voices by converting the voice data; and by the speaker, providing at least two of the modulated plurality of voices to the user.

### Advantageous Effects

In accordance with the present disclosure, by recording user-customized contents and listening to user-customized contents with a user's voice or ideal tone, comfortable and ideal psychological counseling can be conducted. In accordance with the present disclosure, by allowing users to focus more on themselves and think about positive emotions and related experiences, it can help the users with “self-referencing” activities so that the users can focus more on themselves and think about positive emotions and related experiences, and excessive self-immersion for events and experiences related to negative emotions, thereby enabling “self-distancing” that helps to prevent the excessive self-immersion. To implement the above effects, a combination of effective a self-focus shift and the self-talk can be provided.

### BRIEF DESCRIPTION OF THE DRAWINGS

FIG. 1 is a block diagram of a psychology counseling device according to an embodiment of the present disclosure.

FIG. 2 is a flowchart a self-talk according to an embodiment of the present disclosure.

FIGS. 3A-3C are texts based on positive self-talk according to an embodiment of the present disclosure.

FIGS. 4A-4C are texts based on acceptance and commitment therapy according to an embodiment of the present disclosure.

FIGS. 5A-5C are texts based on mindfulness and breathing according to an embodiment of the present disclosure.

FIG. 6 is a flowchart of a method for providing self-talk with an ideal tone according to an embodiment of the present disclosure.

FIGS. 7 and 8 are diagrams for explaining a method of providing an ideal tone according to an embodiment of the present disclosure.

FIGS. 9A-9C show examples of voice expression adjectives for tone control classification according to an embodiment of the present disclosure.

FIGS. 10A-10D are examples of a screen provided to a user according to an embodiment of the present disclosure.

FIGS. 11A-11D are examples of a screen provided to a user according to an embodiment of the present disclosure.

FIG. 12 is a logical tree for explaining a method of providing an adjusted tone according to an embodiment of the present disclosure.

### DESCRIPTION OF THE INVENTION

Hereinafter, with reference to the accompanying drawings, the embodiments of the present disclosure will be described in detail so that those of ordinary skill in the art to which the present disclosure pertains can readily implement them. However, the present disclosure may be implemented in several different forms and is not limited to the embodiments described herein.

In order to clearly explain the present disclosure in the drawings, parts irrelevant to the description are omitted, and similar reference numerals are attached to similar parts throughout the specification.

Throughout the specification, when a part “includes” or “comprises” a certain component, it means that other components may be further included, rather than excluding other components, unless otherwise stated.

It is to be understood that the techniques described in the present disclosure are not intended to be limited to specific embodiments, and include various modifications, equivalents, and/or alternatives of the embodiments of the present disclosure.

The expression “configured to (or set to)” as used in this disclosure, depending on the context, can be used interchangeably with, for example, “suitable for”, “having the capacity to,” “designed to”, “adapted to”, “made to”, or “capable of”. The term “configured (or configured to)” is not necessarily means merely “specifically designed to” hardware. Instead, in some circumstances, the expression “a device configured to” means that the device is “capable of” with other devices or components. For example, the phrases “a processor configured (or configured to perform) A, B, and C,” “a module configured (or configured to perform) A, B, and C”, means a dedicated processor (for example, it may mean an embedded processor) or a generic-purpose processor (e.g., a CPU or an application processor) capable of performing corresponding operations by executing one or more software programs stored in a memory device.

The disclosures in the related art described in the present disclosure are incorporated herein by reference in their entirety, and it will be understood that the contents described in the disclosures in the related art is applied to the portions briefly described in the present disclosure by a person of ordinary skill in the art.

Hereinafter, a psychology counseling device and a method thereof according to an embodiment of the present disclosure will be described with reference to the drawings.

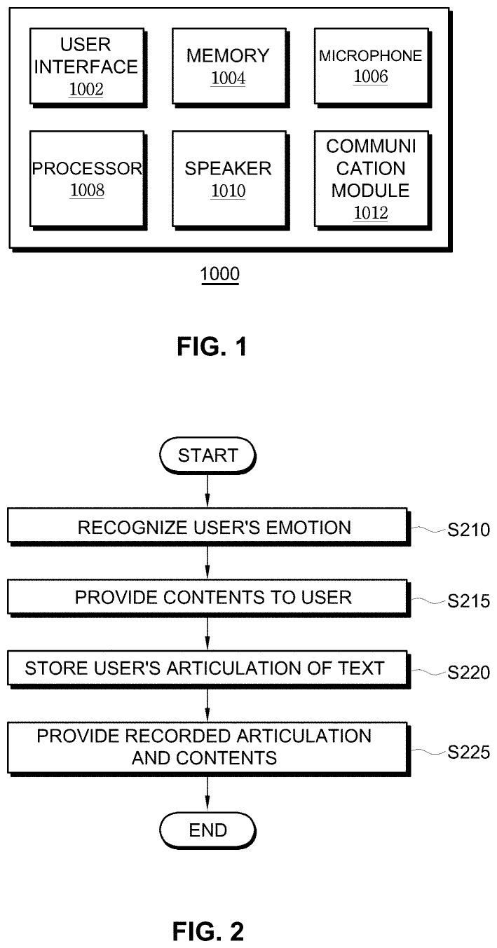

FIG. 1 is a block diagram of a psychology counseling device 1000 according to an embodiment of the present disclosure. The psychology counseling device 1000 may include all kinds of devices which use Internet lines such as an Internet protocol television (IPTV), a smart TV, a connected TV, a set-top box (STB), a smartphone, and a tablet personal computer (PC). The psychology counseling device 1000 may provide a psychological counseling method according to the present disclosure through an application installed in the psychology counseling device 1000.

In an embodiment of the present disclosure, the psychology counseling device 1000 includes a user interface 1002, a memory 1004, a microphone 1006, a processor 1008, a speaker 1010, and a communication module 1012.

The user interface 1002 may provide an interface for providing contents to a user. The user interface 1002 receives an input from a user and provides contents to the user. The user interface 1002 may include a display (not shown). The user interface 1002 may include a touch screen. The psychology counseling device 1000 may output information for executing contents to the user through the user interface 1002. For example, the psychology counseling device 1000 may provide an ecological momentary assessment-based questionnaire survey to figure out a user's emotion through the user interface 1002. In addition, the psychology counseling device 1000 may provide contents for self-talk to the user.

The memory 1004 may be accessed by a computing device and is a computer-readable storage medium such as a data storage device which provides persistent storage of data and executable instructions (e.g., software applications, programs, and functions). Examples of the memory 1004 includes volatile memories, non-volatile memories, fixed and removable media devices, and arbitrarily suitable memory devices or electronic data storages which hold data for a computing device access. Various implemented examples of the memory 1004 may include random access memories (RAMs), read-only memories (ROMs), flash memories, and other types of storage media in various memory device configurations. The memory 1004 is configured to store executable software instructions (e.g., computer executable instructions) executable together with the processor 1008 or a software application which may be implemented as a module.

In an embodiment, the memory 1004 may store instructions for allowing a user to figure out contextual information or to conduct (or assist) a self-talk. The memory 1004 may store information for providing ecological momentary assessment and self-talk. In addition, the memory 1004 may store an instruction required to modulate a received user's voice. For example, modulation of voice may mean changing a pitch of sound, a formant, a speed of voice, a phonatory setting, prosody-intonation, prosodic settings, and articulatory settings and generating a plurality of changed voices of the same text with different voices.

The memory 1004 stores contents to be provided to a user. In an embodiment, the contents may include at least one among texts, background music, and images. For example, the memory 1004 stores texts to be provided to the user and words corresponding to keywords of the texts. In an embodiment, the keywords and the content, which are to be provided to the user, may be stored in pairs. For example, when words of negative emotion and contents (texts) of negative emotion are paired and stored in the memory 1004, and when the user selects a word of negative emotion, the paired content (text) of negative emotion may be provided to the user through the interface 1002.

In an embodiment, the contents may include texts based on positive self-talk (PST), texts based on concepts of forgiveness, acceptance, self-respect or other respect, gratitude, self-compassion, love-kindness, and texts based on acceptance and commitment therapy among cognitive behavior therapy (CBT) methods of treating anxiety and depressive disorders.

The microphone 1006 may receive a user's voice. The user may record the sentences provided by the psychology counseling device 1000 through the microphone 156. The psychology counseling device 1000 may collect the user's voice through the microphone 1006 and analyze the user's voice to confirm user's intent and emotion. For example, the user may articulate the texts provided through the user interface 1002. The microphone 1006 may recognize the user's articulation, and the psychology counseling device 1000 may store the user's articulation in the memory 1004.

The processor 1008 may include an integrated circuit, a programmable logic device, a logic device formed using one or more semiconductors, a processor formed as a system-on-chip (SoC), and components of other implementations of silicon and/or hardware such as a memory system. The processor 1008 may be configured to analyze a voice stored in the memory 1004. In addition, the processor 1008 may be configured to control components of the psychology counseling device 1000 and provide information stored in the memory 1004 to the user or analyze the information stored in the memory 1004.

The psychology counseling device 1000 may further include any type of system bus which combines various components in the psychology counseling device 1000 or other data and an instruction convey system. The system bus may include control and data lines as well as any one or a combination of different bus structures and architectures.

The speaker 1010 conveys contents to the user as auditory information. In an embodiment, the speaker 1010 may convey a sentence recorded by the user to the user as the voice recorded by the user and a sound modulated from the user's voice.

The communication module 1012 is configured such that the psychology counseling device 1000 communicates with an external device to receive information. A communication method of the communication module 1012 may employ a network established according to global system for mobile communication (GSM), code division multi access (CDMA), high speed downlink packet access (HSDPA), high speed uplink packet access (HSUPA), long term evolution (LTE), LTE-advanced (LTE-A), wireless local area network (WLAN), wireless-fidelity (Wi-Fi), Wi-Fi direct, digital living network alliance (DLNA), wireless broadband (WiBro), and world interoperability for microwave access (WiMAX), but the present disclosure is not limited thereto, and the communication method may include all transmission method standards to be developed in the future. The communication method may include all method capable of transmitting and receiving data through wired and wireless manners. The contents stored in the memory may be updated through the communication module 1012.

The psychology counseling device 1000 is configured to convert a voice into a sentence (Speech To Text (STT)) and a sentence into voice (Text To Speech (TTS)). The functions of STT and TTS are basically provided by smart devices, and thus detailed descriptions therefor are omitted herein.

The psychology counseling device 1000 may be configured to implement an artificial intelligence model. The artificial intelligence model of the present disclosure is configured to perform natural language processing on a user's articulation. As described below, the artificial intelligence model may be an artificial intelligence model obtained by training a learning model including an artificial neural network (ANN). For example, a natural language processor may include bidirectional encoder representation from transformers (BERT) of Google and an applied model therefor, a generative pre-training (GPT) and an applied model therefor, XLNET, RoBERTa, and ALBERT. In the present disclosure, the artificial intelligence model may train a learning model including an ANN through a large amount of learning data to optimize parameters inside the ANN and may obtain a response with respect to a new input using the trained learning model. Examples of the ANN include at least one among a convolutional neural network (CNN), a deep neural network (DNN), a recurrent neural network (RNN), a restricted Boltzmann machine (RBM), a deep belief network (DBN), a bidirectional recurrent deep neural network (BRDNN), and deep Q-networks or a combination therefor, but the present disclosure is not limited thereto.

FIG. 2 is a flowchart a self-talk according to an embodiment of the present disclosure. The psychology counseling device 1000 recognizes a user's emotion (S210). In an embodiment, the psychology counseling device 1000 may recognize the user's emotion through the user interface 1002. The psychology counseling device 1000 may display an indicator (e.g., an icon, a character, or the like) indicating a person's emotion through the user interface 1002. For example, the psychology counseling device 1000 may display icons or words indicating fear, anger, happiness, joy, sadness, depression, anxiety, or positive, neutral, and negative with respect to these emotions.

In an embodiment, the emotion may be presented in a plurality of different colors and icons. For example, the emotion may be presented as seven icons of different colors and expressions. Two icons “Smiley and yellow” and “Happy and green” which are connected to a positive content, three icons “Depressed and blue”, “Sad and purple”, and “Angry and red” which are connected to a negative content, and two icons “Distracted and sky-blue” and “Neutral and orange” which are connected to a neutral content are provided. The psychology counseling device 1000 may recognize an emotional state of the user based on the emotion icon or word selected by the user. In an embodiment, recognizing the emotional state of the user by the psychology counseling device 1000 does not mean that machines understand human emotions, but means that the psychology counseling device 1000 confirms a current state of the user from a user's input, and may include a preparation step of selecting texts to be provided to the user by determining whether the user's emotional state corresponds to one of a plurality of pre-stored states.

The psychology counseling device 1000 may provide a question to the user through the user interface 1002, receive a response thereto, and recognize the emotional state of the user from the response. In an embodiment, the question-response may be a momentary assessment question-response. An example of a momentary assessment question of “How are you feeling now? Choose the emotion you feel right now.” is questioned, and then the user answers a feeling in response to the question so that an emotional state is recognized.

In an embodiment, the psychology counseling device 1000 may recognize the emotional state of the user through voice information of the user. For example, an arbitrary sentence is provided to the user, and in response to that the user articulates the arbitrary sentence, the psychology counseling device 1000 may recognize the emotional state of the user from voice information of the articulation. As an example, the emotional state of the user may be recognized based on a tremor of the voice, a change and a degree of the change in an intensity of the voice, and a time taken to answer a question. It is to be understood that a method of recognizing an emotional state from the voice information may be performed based on the contents disclosed in a document described as the related art document herein.

The psychology counseling device 1000 provides contents to the user (S215). In an embodiment, the contents may include at least one among texts, images and background music. The psychology counseling device 1000 may provide the contents to the user through the user interface 1002. In an embodiment, the psychology counseling device 1000 may provide texts based on the user's emotion recognized in operation S210. For example, the psychology counseling device 1000 may provide different texts based on happiness, joy, sadness, depression, anxiety, or positive, neutral, and negative with respect to these emotions.

That is, the psychology counseling device 1000 is configured to store contents corresponding to each icon corresponding to the user's emotion in the memory 1004 and is configured to recognize the user's emotion (for example, by receiving an input that the user selects an icon) to provide the user with the contents corresponding to each icon corresponding to the user's emotion.

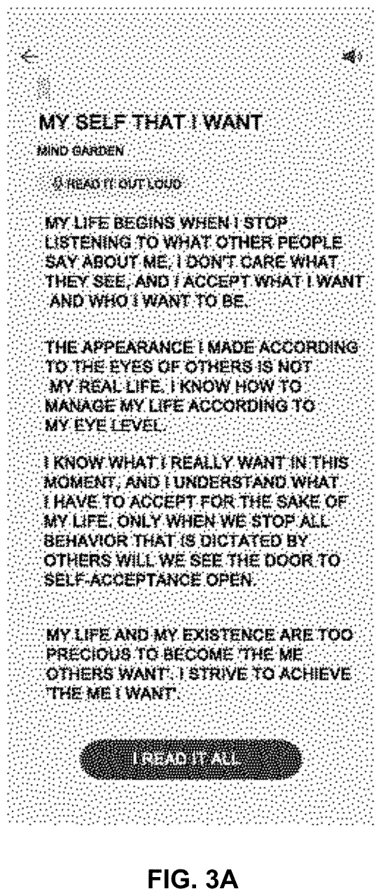

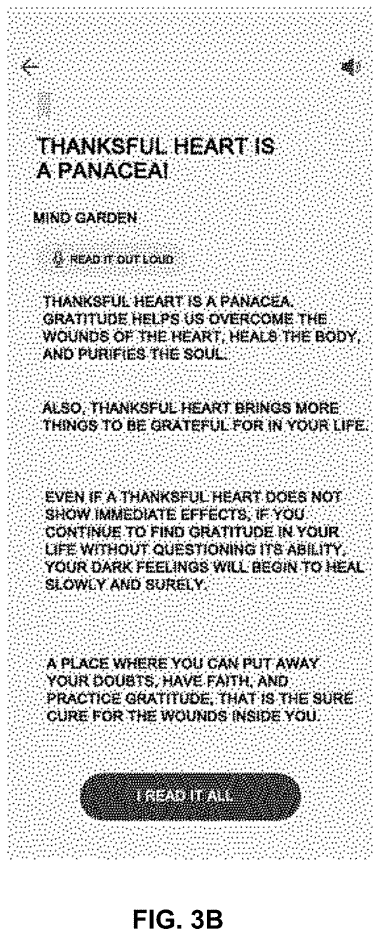

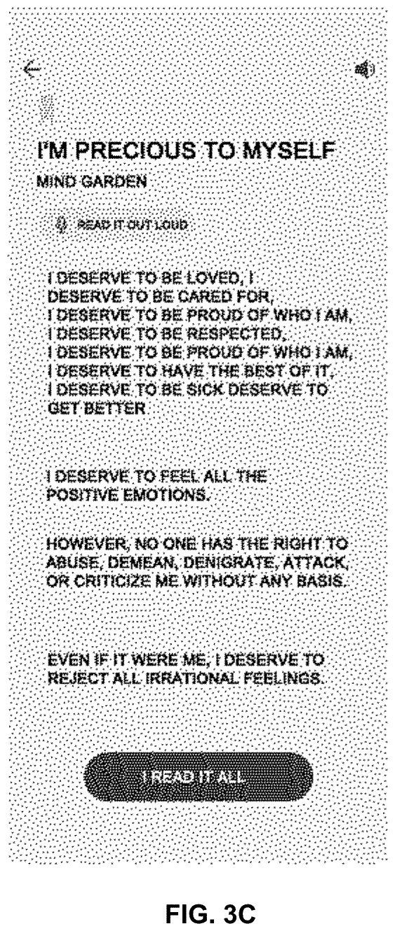

In an embodiment, in response to recognizing that the user's emotion is positive, the psychology counseling device 1000 may provide texts based on PST. For example, the PST may include articulation which allows encourages the user to have a positive feeling about himself or herself and encourages the user. A positive text based on the PST is provided as a text organized based on concepts of forgiveness, acceptance, other respect, gratitude, self-compassion, and love-kindness which are regarded to be effective for depression and anxiety in positive psychology. FIGS. 3A-3C are texts based on positive self-talk according to an embodiment of the present disclosure.

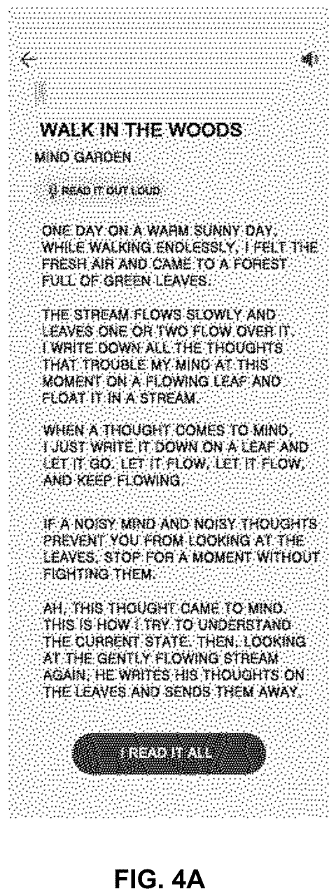

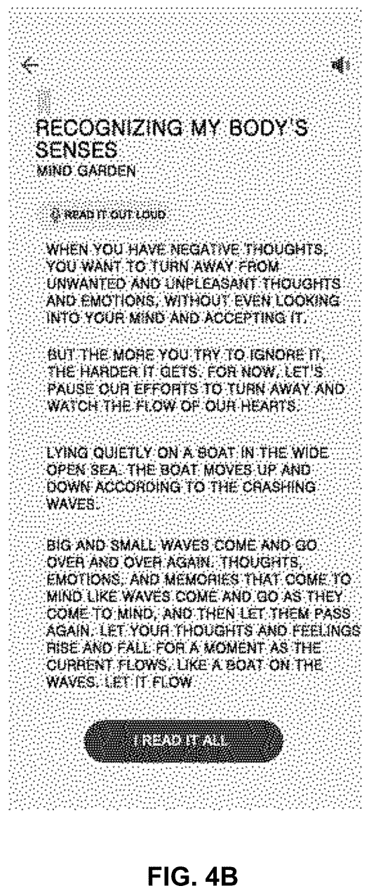

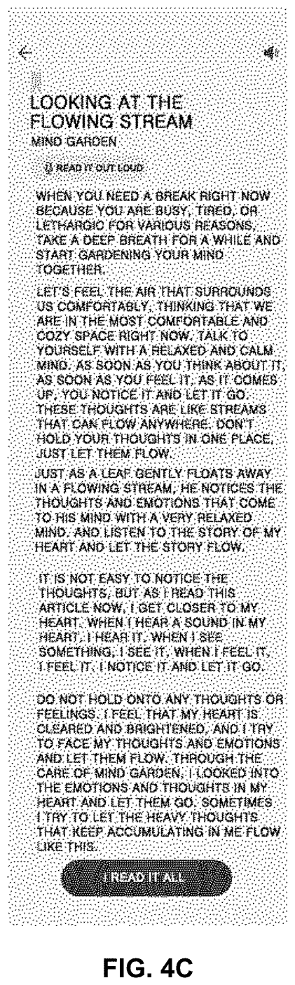

In response to recognizing that the user's emotion is negative, the psychology counseling device 1000 may provide texts based on an acceptance and commitment therapy (ACT) among CBT methods of treating anxiety and depressive disorders. The ACT consists of contents of acceptance, cognitive defusion, self as context, being present, value, and committed action, conveys the contents in a metaphorical way, and provides contents capable of being applied to real life. FIGS. 4A-4C are texts based on acceptance and commitment therapy according to an embodiment of the present disclosure.

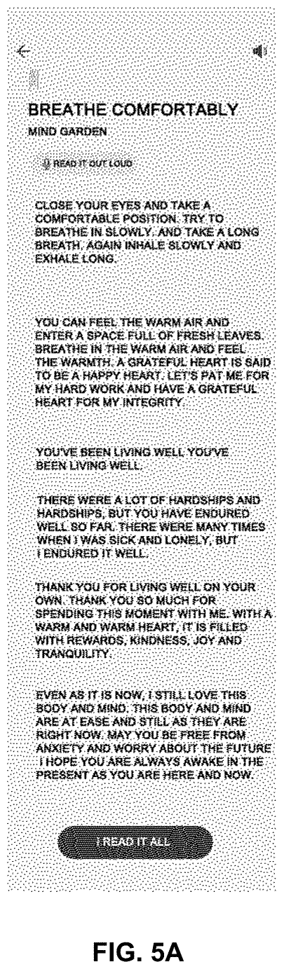

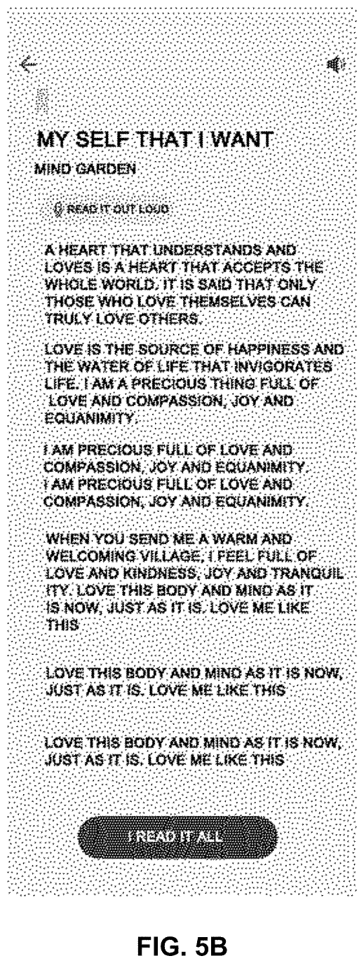

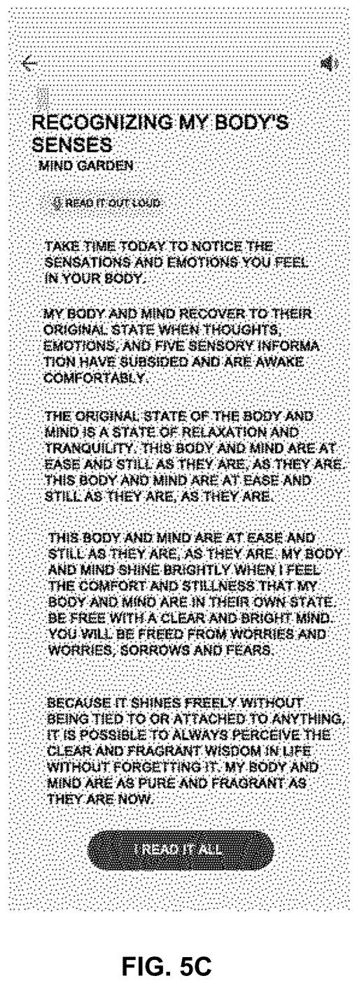

In response to recognizing that the user's emotion is a neutral emotion, the psychology counseling device 1000 may provide the user with texts based on mindfulness and breathing. FIGS. 5A-5C are texts based on mindfulness and breathing according to an embodiment of the present disclosure.

In an embodiment, the providing of the texts to the user may be performed through an artificial intelligence model. The artificial intelligence model may be trained with icons, texts, voices, and question-answers as inputs and with outputs with respect to the inputs as texts. That is, the result may be classified for a combination of at least one of a text, an icon, selected by the user, a user's voice, and a user's response to a question, and a text suitable for the classification may be provided to the user.

The psychology counseling device 1000 stores the user's articulation corresponding to the text in the memory 1004 (S220). The user recognizes and reads (articulates) the provided text. In an embodiment, the psychology counseling device 1000 determines whether a keyword of the text is present during the user's articulation. In response to determining that the keyword of the text is present in the user's articulation, the psychology counseling device 1000 stores the entirety of the user's articulation.

In an embodiment, the psychology counseling device 1000 may recognize and store the user's articulation without pressing a separate record button by the user. For example, the psychology counseling device 1000 may automatically initiate a function of automatically storing the user's speech at the same time as providing the text to the user or after a predetermined time after providing the text. Thus, the psychology counseling device 1000 may store the user's articulation by simply articulating the text provided in the user interface 1002 without pressing a separate record button by the user.

The psychology counseling device 1000 provides the recorded articulation and the contents to the user (S225). In an embodiment, the articulated contents may be provided as texts.

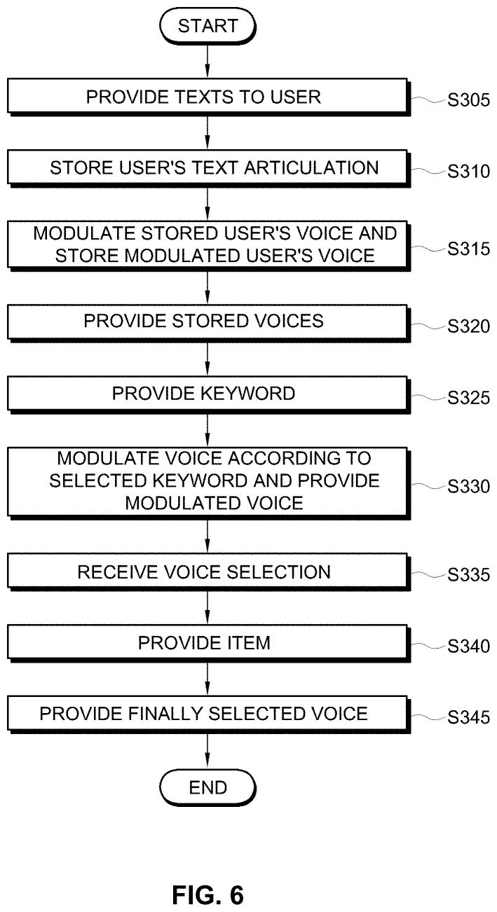

FIG. 6 is a flowchart of a method for providing self-talk with an ideal tone according to an embodiment of the present disclosure.

Referring to FIG. 6, the psychology counseling device 1000 provides texts to the user (S305). In an embodiment, the providing of the texts may be performed similarly to the providing of the texts in operation S215 of FIG. 2. That is, the psychology counseling device 1000 may recognize the user's emotion and provide the texts in response thereto. Alternatively, the psychology counseling device 1000 may provide a text determined according to a menu selection of the user. For example, a menu provided to the user by the psychology counseling device 1000 may include “tone control,” “experience course,” and the like and provide texts in response to the user selecting the menu.

The psychology counseling device 1000 stores a user's text articulation (S310). The user's articulation may be stored in the form of voice data. The user recognizes and reads (articulates) the provided text. In an embodiment, the psychology counseling device 1000 determines whether a keyword of the text is present during the user's articulation. In response to determining that the keyword of the text is present in the user's articulation, the psychology counseling device 1000 stores the entirety of the user's articulation.

The psychology counseling device 1000 stores the stored user's voice and a voice obtained by modulating the user's voice (S315). The psychology counseling device 1000 stores the stored user's voice and N voices obtained by modulating the user's voice.

As for a human voice, the transmission path is different between the sound heard by speaking by the person and the sound recorded and heard. This is because the sound from the vocal cords is conveyed directly through the bones and muscles to the inner ear, but the recorded voice is produced as air from the lungs passes through the vocal cords in the larynx. Accordingly, the person feels that the sound he or she hears while speaking is different from the sound he or she hears on a recording. More specifically, when his or her voice is conveyed directly to the inner ear, a low-tone sound is emphasized, whereas a mid-tone and a high-tone tend to be emphasized in a sound produced through a vibration of the vocal cords. When a psychological counseling is performed through self-talk, the user may feel awkward because the recorded voice is heard to the user.

In an embodiment, the psychology counseling device 1000 of the present disclosure may modulate the stored voice to resemble the sound conveyed directly to the inner ear by his or her voice. In addition, the psychology counseling device 1000 may modulate the user's voice in various ways.

In an embodiment, the psychology counseling device 1000 may extract and modulate features such as pitches, characteristic waveforms, and formants from the stored voice data to store the voice data in which the user's voice is modulated. Accordingly, the articulation of the same text may be stored as a plurality of different voices. When the voice data is stored, the psychology counseling device 1000 may increase or decrease at least one among the pitches, the waveforms, and the formants extracted from the voice data and automatically modulate the user's voice. In this case, an amount in which at least one among the pitches, the waveforms, and the formants may be determined in advance by a rule and may be stored in the memory 1004 of the psychology counseling device 1000. In an embodiment, based on the recorded voice (raw voice), the pitch is adjusted by +2 or −2 and the formant is adjusted by +1 or −1 so that, for example, fourteen tone-modulated types excluding the recorded voice (raw voice) may be generated. The pitch is a term referring to a level of a sound and physically means a difference in frequency, and the higher the frequency, the higher the pitch. In an embodiment, the pitch may be adjusted in units of 1 Hz, 2 Hz, 3 Hz, or 4 Hz. The unit for adjusting the pitch may be freely set. When a person makes a voice, a frequency resonates and an amplitude of the frequency is increased, and in this case, the formant means a frequency amplitude or a frequency band where the resonance occurs. The formant adjustment may mean adjusting or moving an amplitude or a band of the resonant frequency.

The rule regarding the modulation may be determined through repeated tests. For example, an administrator of the psychology counseling device 1000 may obtain a desired modulated voice by storing the user's voice data and performing a test for modulating the user's voice data and may determine a modulation rule for obtaining a desired modulated voice.

The memory 1004 of the psychology counseling device 1000 may store a plurality of predetermined rules which modulate the user's voice. The psychology counseling device 1000 may automatically modulate and store the stored user's voice data according to the above rules. Each of the plurality of predetermined rules may be stored corresponding to a keyword. Accordingly, when the user selects a specific keyword, the counseling device 1000 may play a voice modulated according to a rule corresponding to the selected keyword to the user.

In another embodiment, the user may input an input for modulating at least one among a pitch, a waveform, and a formant through the user interface 1002 of the psychology counseling device 1000, and thus the stored voice data may be modulated. That is, the user may manually set and store a desired modulated voice.

In another embodiment, the memory 1004 of the psychology counseling device 1000 may store a plurality of predetermined rules which modulate the user's voice. Each of the plurality of predetermined rules may be stored corresponding to a keyword. When the user selects a specific keyword, the counseling device 1000 may modulate voice data according to a rule corresponding to the selected keyword and play the modulated voice data to the user.

In still another embodiment, a sample voice may be stored in the memory 1004 of the psychology counseling device 1000. The sample voice includes voices stored by articulating the same articulation into a plurality of different voices. The user may listen to the sample voice and select a voice similar to a desired voice. The psychology counseling device 1000 may modulate the stored voice data articulated by the user to allow the user's voice to be similar to a voice selected by the user.

The psychology counseling device 1000 provides the recorded articulation to the user (S320). In an embodiment, the psychology counseling device 1000 provides a plurality of stored voices and a plurality of modulated voices (S320). For example, the psychology counseling device 1000 may provide the user with four different voices. The user may listen to the plurality of voices and select a desired voice.

The psychology counseling device 1000 may provide a keyword to the user (S325). The keyword may include a keyword indicating an emotion. The user may select a desired keyword.

In response to the keyword selected by the user, the psychology counseling device 1000 may re-modulate the voice selected in operation S320 and provide the re-modulated voice to the user (S330). For example, since the rules for modulating voice data corresponding to keywords are stored in the memory 1004 of the psychology counseling device 1000, the psychology counseling device 1000 may retrieve a rule for modulating voice data based on the selected keyword from the memory 1004 and modulate the voice data based on the retrieved rule. The psychology counseling device 1000 may provide re-modulated voices to the user. The user may select a desired voice based on the provided voices.

The psychology counseling device 1000 receives a voice selection of the user (S335). In response to receiving the voice selection of the user, the psychology counseling device 1000 provides an item to the user (S340). The item may be used to decorate a part of a screen output from the user interface 1002. The providing of the item may be omitted.

In response to receiving the voice selection of the user, the psychology counseling device 1000 provides the finally selected voice to the user (S345).

In an embodiment, the operations of FIGS. 5 and 6 may be provided in combination.

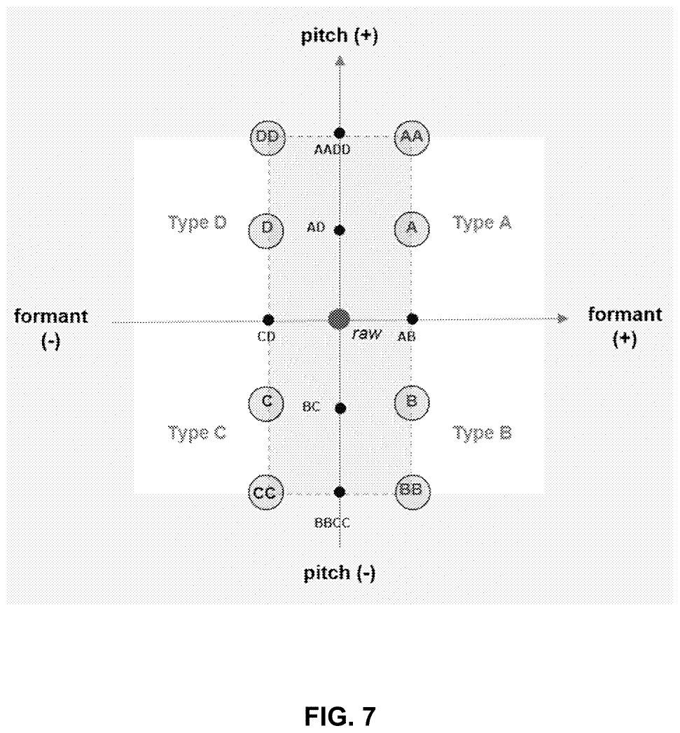

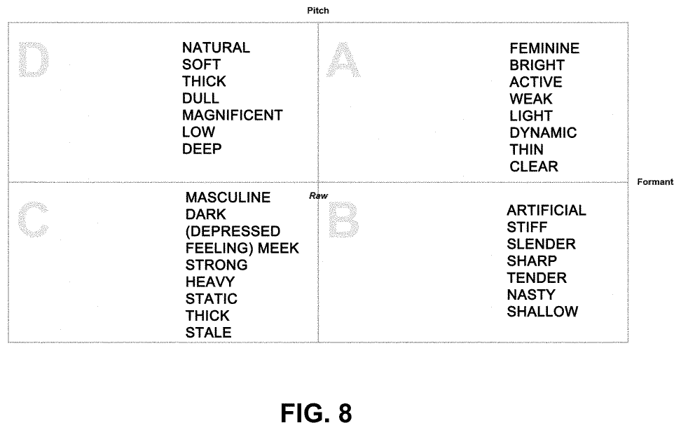

FIGS. 7 and 8 are diagrams for explaining a method of providing an ideal tone according to an embodiment of the present disclosure.

Referring to FIG. 7, a center point (indicated as a “raw”) at which a formant axis (x axis) and a pitch axis (y axis) intersect is a user's tone with no change. A tone in which the formant and the pitch are increased is referred to as type A (first quadrant), a tone in which the formant is increased and the pitch is decreased is referred to as type B (fourth quadrant), a tone in which the formant and the pitch are decreased is referred to as type C (third quadrant), and a tone in which the formant is decreased and the pitch is increased is referred to as type D (second quadrant). In an embodiment, as shown in FIG. 7, according to a degree of adjustment of the formant and the pitch, fourteen tones, in which formants and pitches are adjusted, of A, AB, AA, AD, AADD, B, BB, BC, BBCC, C, CC, CD, D, and DD may be provided. The fourteen tones are exemplary numbers, and it is to be understood that various numbers of tones may be generated and provided according to adjustment of the formant and the pitch. The pitch is a term referring to a level of a sound and physically means a difference in frequency, and the higher the frequency, the higher the pitch. In an embodiment, the pitch may be adjusted in units of 1 Hz, 2 Hz, 3 Hz, or 4 Hz. The unit for adjusting the pitch may be freely set. When a person makes a voice, a frequency resonates and an amplitude of the frequency is increased, and in this case, the formant means an amplitude or a band of the resonant frequency. The formant adjustment may mean adjusting or moving an amplitude or a band of the resonant frequency. In an embodiment, the pitch may be adjusted in units of 1 Hz, 2 Hz, 3 Hz, or 4 Hz.

In an embodiment, as shown in FIG. 7, the psychology counseling device 1000 may provide the user with a screen for adjusting a tone with the formant and the pitch as respective axes based on a user's original voice, and the user may select a formant and a pitch. For example, the user may select a desired point through the user interface 1002, for example, a touch screen. The psychology counseling device 1000 may receive a selection of the user and adjust the tone of the user's voice.

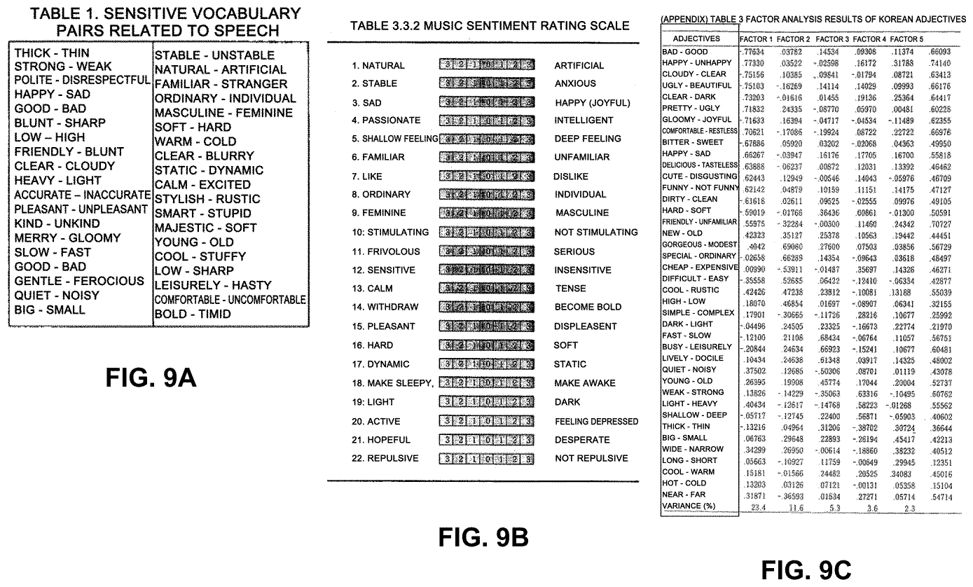

Referring to FIG. 8, adjectives (or keywords) corresponding to type A, type B, type C, and type D are disclosed. As shown in FIGS. 9A-9C, these adjectives are extracted with reference to Documents entitled “Study on Auditory Emotion Measurement Technology and DB Development, 1998” by Jin-Hoon Son, “Semantic Structure of Korean Adjectives for Emotion Measurement. Emotional Science, 1(2), 1-11, 1998” by Mi-Ja Park, Su-Gil Shin, Kwang-Hee Han, and Sang-Min Hwang, and “A study on pleasant voice synthesized sounds using emotional evaluation, Journal of the Korean Society of Ergonomics, 21(1), 51-65, 2002” by Yong-guk Park, Jae-guk Kim, Yong-woong Jeon, and Am Jo. In an embodiment, the psychology counseling device 1000 may provide the user with the adjectives shown in FIG. 8 and adjust the tone of the user's voice by receiving an input of the adjective selected by the user. The adjectives corresponding to type A, type B, type C, and type D may be stored in the memory 1004 of the psychology counseling device 1000. In addition, the degree of adjusting the tone according to each adjective, for example, the degrees of adjusting the pitch and the formant may be matched and stored in the memory 1004.

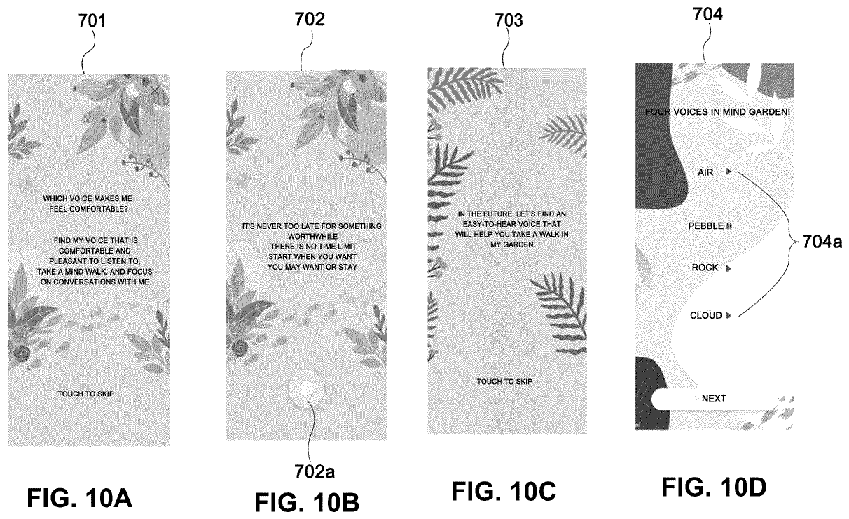

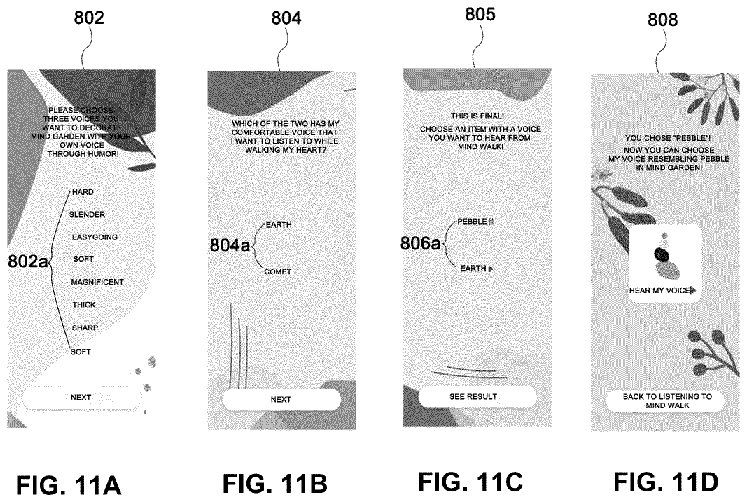

FIGS. 10A-10D and FIGS. 11A-11D are examples of a screen provided to a user according to an embodiment of the present disclosure. Referring to FIGS. 10A-10D, the user is provided with contents (e.g., texts) provided through the screen 701, 702, 703, 704 provided by the user interface 1002. The user may store an articulated text using a recording button 702a. The psychology counseling device 1000 provides words 704a corresponding to type A, type B, type C, and type D to the user through a screen 704. When the user selects any one among the words 704a, the user's articulation may be provided to the user with a tone in which the pitch and the formant corresponding to Type A, Type B, Type C, or Type D are adjusted. For example, in response to the user selecting a word corresponding to type A, the pitch and the formant of the user's articulation stored to correspond to A or AA of FIG. 7 may be adjusted. In response to the user selecting a word corresponding to type B, the pitch and the formant of the user's articulation stored to correspond to B or BB of FIG. 7 may be adjusted. In response to the user selecting a word corresponding to type C, the pitch and the formant of the user's articulation stored to correspond to C or CC of FIG. 7 may be adjusted. In response to the user selecting a word corresponding to type D, the pitch and the formant of the user's articulation stored to correspond to D or DD of FIG. 7 may be adjusted.

Referring to FIGS. 11A-11D, the user receives adjectives or keywords 802a indicating a voice so as to perform a more subdivided tone adjustment of his/her preferred voice through a screen 802 provided by the user interface 1002. That is, the psychology counseling device 1000 may retrieve the adjectives stored in the memory 1004 and provide the retrieved adjectives to the user through the user interface 1002. As shown in FIG. 8, each adjective belongs to one of types A to D. The user may select adjectives, for example, three adjectives. A tone (e.g., a pitch and a formant) of the user's articulation may be adjusted according to the adjective selected by the user. The psychology counseling device 1000 may recognize an orientation (or tendency) of a voice desired by the user based on the adjective selected by the user.

The user may receive two tone-adjusted voice types 804a corresponding to more selected orientations (or tendencies) through the screen 804 provided by the user interface 1002. For example, when the user prefers type A in orientation B, tone-adjusted voice types of A and AB in orientation B, excluding AA in a type A category, is provided so that the user may select a tone. The user may select any one of the two tone-adjusted voice types 804a.

The user may receive an initially selected type and a detailed tone-adjusted type 806a through a screen 805 provided by the user interface 1002. The user may select any one of the initially selected type and the detailed tone-adjusted type 806a. In another embodiment, together with the initially selected type and the detailed tone-adjusted type 806a, the psychology counseling device 1000 may provide the user with a voice initially articulated by the user without tone adjustment.

The psychology counseling device 1000 provides a screen 808 which provides the finally selected voice to the user.

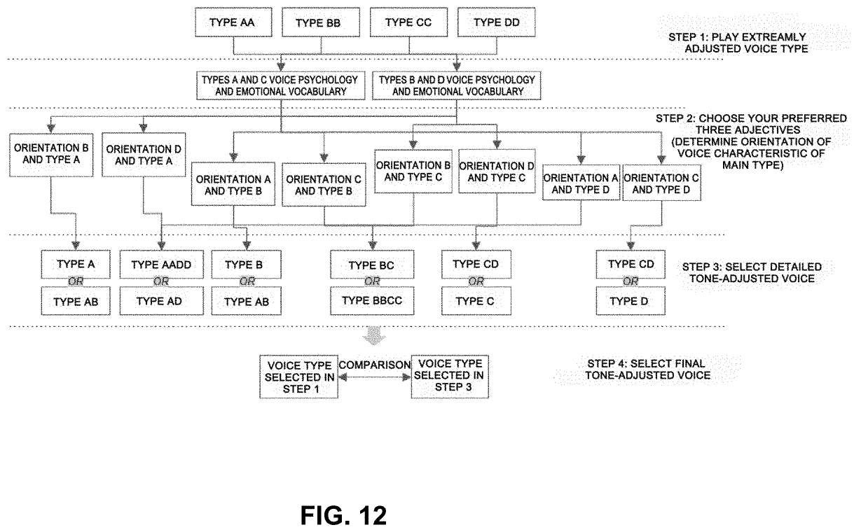

FIG. 12 is a logical tree for explaining a method of providing an adjusted tone according to an embodiment of the present disclosure. A method of providing an adjusted tone will be described with reference to FIGS. 10A-10D, 11A-11D, and 12. The psychology counseling device 1000 provides words 704a corresponding to type A, type B, type C, and type D to the user through a screen 704. When the user selects any one among the words 704a, the user's articulation may be provided to the user with a tone in which the pitch and the formant corresponding to Type A, Type B, Type C, or Type D are extremely adjusted. In an embodiment, when the user selects a word corresponding to type A, a pitch and a formant may be adjusted to correspond to AA of FIG. 7, and when the user selects a word corresponding to type B, a pitch and a formant may be adjusted to correspond to BB of FIG. 7, and when the user selects a word corresponding to type C, a pitch and a formant may be adjusted to correspond to CC of FIG. 7, and when the user selects a word corresponding to type D, a pitch and a formant may be adjusted to correspond to DD of FIG. 7. The psychology counseling device 1000 may provide the user with the user's articulation adjusted to type AA, type BB, type CC, or type DD (Step 1).

As disclosed in the screen 802 of FIGS. 11A-11D, the psychology counseling device 1000 may retrieve the adjectives 802a corresponding to types A to D from the memory 1004 and provide the retrieved adjectives 802a to the user through the user interface 1002. In an embodiment, as shown in FIG. 7, among four types A, B, C, and D selected by the user, adjectives of adjacent types may be provided to the user. The user may select adjectives, for example, three adjectives. The psychology counseling device 1000 may recognize an orientation (or tendency) of a voice desired by the user based on the adjective selected by the user. For example, in response to the user selecting a voice of type AA, adjectives for adjacent types B and D in the quadrants among voices of type A may be provided. From the selection of the user, it is possible to recognize whether the user prefers a voice of side B in type A (type A in orientation B), or a voice of side D in type A (type A in orientation D) (Step 2).

The psychology counseling device 1000 may provide the two tone-adjusted voices 804a corresponding to an orientation of the tendency that the user selects more among the selected adjectives. For example, when the user prefers type A in orientation B, tone-adjusted voice types of A and AB in orientation B, excluding AA in a type A category, may be provided. The user may select any one of the two voices 804a (Step 3).

The psychology counseling device 1000 may provide the initially selected type (e.g., AA, BB, CC, or DD) and the detailed tone-adjusted types 806a through the screen 805 provided by the user interface 1002. The user may select any one of the initially selected type and the detailed tone-adjusted type 806a (Step 4).

The device and method described above may be implemented as a hardware component, a software component, and/or a combination of the hardware component and the software component. For example, devices and components described in the embodiments may be implemented using one or more general purpose computers or special purpose computers, for example, a processor, controller, arithmetic logic unit (ALU), digital signal processor, microcomputer, field programmable array (FPA), programmable logic unit (PLU), microprocessor, or a certain other device capable of executing and responding to instructions. The processing device may execute an operating system (OS) and one or more software applications running on the operating system. The processing device may also access, store, manipulate, process, and generate data in response to execution of the software. Although, for the convenience of understanding, there are instances where one processing device is described as being used, a person of ordinary skill in the art will recognize that a processing device may include a plurality of processing elements and/or a plurality of types of processing elements. For example, the processing device may include a plurality of processors or one processor and one controller. Other processing configurations are also possible, such as parallel processors.

Software may include a computer program, code, instructions, or a combination of one or more of these, and configure a processing unit to behave as desired, or independently or collectively give instructions to the processing unit. The software and/or data may be permanently or temporarily embodied on a certain machine, component, physical device, virtual equipment, computer storage medium or device, or transmitted signal wave in order to be interpreted by or to provide instructions or data to the processor. The software may be distributed over networked computer systems and stored or executed in a distributed manner. The software and data may be stored in one or more computer-readable recording media.

The described embodiments of the present disclosure also allow certain tasks to be performed on a distributing computing environment performed by remote processing devices that are linked through a communications network. In the distributed computing environment, program modules may be located in both local and remote memory storage devices.

As described above, although the embodiments have been described with reference to the limited drawings, those of ordinary skill in the art may apply various technical modifications and variations to the above, based on them. Appropriate results can be achieved when, for example, the described techniques are performed in an order different from the described method, and/or the described components of a system, structure, device, circuit, etc. are combined or combined in a different form than the described method, or other components or an equivalent may be substituted or exchanged to achieve an appropriate result.

Therefore, other implementations, other embodiments, and equivalents to the claims are also within the scope of the following claims.

What is claimed is:

1. A psychology counseling device comprising:
a user interface configured to receive an input from a user and provide information;
a microphone configured to collect a voice of the user;
a speaker configured to convey auditory information to the user;
a processor configured to control the user interface, the microphone, and the speaker; and
a memory accessible by the processor and configured to store executable instructions,
wherein the memory is configured to further store texts to be provided to the user and voice data received from the user, and
the executable instructions, when executed by the processor, causes the processor to perform:
recognizing an emotional state of the user based on the user's input;
providing texts including different contents to the user according to the emotional state of the user;
receiving a voice that the user articulates the texts and storing the voice in the memory as the voice data;
obtaining a plurality of modulated voices by converting the voice data; and
providing at least two among the plurality of modulated voices to the user.

2. The psychology counseling device of claim 1, wherein
the recognizing of the emotional state of the user based on the user's input includes:
providing icons or words indicating the emotional state to the user; and
receiving an icon or a word selected by the user.

3. The psychology counseling device of claim 2, wherein:
the icon or the word indicating of the emotional state is an icon or a word indicating positive, negative, or neutral; and
the providing of the texts including the different contents to the user according to the emotional state of the user includes at least one among:
in response to the emotional state of the user being positive, providing a text based on a positive self-talk (PST);
in response to the emotional state of the user being negative, providing a text based on a cognitive behavioral therapy (CBT) method; and
in response to the emotional state of the user being neutral, providing a text based on mindfulness and breathing.

4. The psychology counseling device of claim 1, wherein:
the recognizing of the emotional state of the user based on the user's input includes:
providing a momentary assessment question to the user;
receiving a response to the momentary assessment question from the user; and
recognizing the emotional state based on the question and the response.

5. The psychology counseling device of claim 1, wherein the obtaining of the plurality of modulated voices by converting the voice data includes:
automatically performing the obtaining of the plurality of modulated voices according to a predetermined rule in response to the user receiving the voice that the user articulates the texts and storing the voice as voice data.

6. The psychology counseling device of claim 5, wherein the automatically performing of the obtaining of the plurality of modulated voices according to a predetermined rule in response to the user receiving the voice that the user articulates the texts and storing the voice as voice data includes:
providing the user with a modulated voice of a first type in which both a pitch and a formant of the voice data are increased, a modulated voice of a second type in which the pitch of the voice data is decreased and the formant of the voice data is increased, a modulated voice of a third type in which the pitch and the formant of the voice data are decreased, and a modulated voice of a fourth type in which the pitch of the voice data is increased and the formant of the voice data is decreased.

7. The psychology counseling device of claim 1, wherein the obtaining of the plurality of modulated voices by converting the voice data includes:
providing the user with a modulated voice of a first type in which both a pitch and a formant of the voice data are increased, a modulated voice of a second type in which the pitch of the voice data is decreased and the formant of the voice data is increased, a modulated voice of a third type in which the pitch and the formant of the voice data are decreased, and a modulated voice of a fourth type in which the pitch of the voice data is increased and the formant of the voice data is decreased;
receiving any one of the first to fourth types selected by the user;
determining which element of the pitch and the formant the user wants to adjust based on the type selected by the user, which includes providing keywords corresponding to the first to fourth types to the user; and
in response to receiving the keyword selected by the user, adjusting the pitch and the formant of the voice data and providing the adjusted voice data to the user.

8. The psychology counseling device of claim 1, wherein the providing of at least two of the plurality of modulated voices to the user includes:
further providing the user with the voice that the user articulates the texts.

9. The psychology counseling device of claim 1, wherein the texts provided to the user includes any one of a text based on a positive self-talk (PST), a text based on a cognitive behavioral therapy (CBT) method, and a text based on mindfulness and breathing.

10. A psychology counseling method of providing self-talk to a user using a psychological counseling device including a user interface, a memory, a microphone, a speaker, and a processor configured to control the user interface, the memory, the microphone, and the speaker, the psychological counseling method comprising:
recognizing, by the user interface, an emotional state of the user based on the user's input;
retrieving texts including different contents from the memory according to the emotional state of the user and providing the texts to the user;
receiving a voice that the user articulates the texts through the microphone and storing the voice in the memory as the voice data;
obtaining, by processor, the a plurality of modulated voices by converting the voice data; and
providing, by the speaker, at least two among the plurality of modulated voices to the user.

11. The psychology counseling method of claim 10, wherein the recognizing of the emotional state of the user based on the user's input includes:
providing icons or words indicating the emotional state to the user; and
receiving an icon or a word selected by the user.

12. The psychology counseling method of claim 11, wherein:
the icon or the word indicating the emotional state is an icon or a word indicating positive, negative, or neutral; and
the retrieving of the texts including different contents from the memory according to the emotional state of the user and providing the texts to the user includes at least one among:
in response to the emotional state of the user being positive, providing a text based on a positive self-talk (PST);
in response to the emotional state of the user being negative, providing a text based on a cognitive behavioral therapy (CBT) method; and
in response to the emotional state of the user being neutral, providing a text based on mindfulness and breathing.

13. The psychology counseling method of claim 11, wherein the recognizing of the emotional state of the user based on the user's input includes:
providing, by processor, the a momentary assessment question to the user;
receiving a response to the momentary assessment question from the user; and
recognizing the emotional state based on the question and the response.

14. The psychology counseling method of claim 10, wherein the obtaining of the plurality of modulated voices by converting the voice data includes:
automatically performing the obtaining of the plurality of modulated voices according to a predetermined rule in response to the user receiving the voice that the user articulates the texts and storing the voice as voice data.

15. The psychology counseling method of claim 14, wherein the automatically performing of the obtaining of the plurality of modulated voices according to a predetermined rule in response to the user receiving the voice that the user articulates the texts and storing the voice as voice data includes:
providing the user with a modulated voice of a first type in which both a pitch and a formant of the voice data are increased, a modulated voice of a second type in which the pitch of the voice data is decreased and the formant of the voice data is increased, a modulated voice of a third type in which the pitch and the formant of the voice data are decreased, and a modulated voice of a fourth type in which the pitch of the voice data is increased and the formant of the voice data is decreased.

16. The psychology counseling method of claim 10, wherein the obtaining of the plurality of modulated voices by converting the voice data includes:
providing the user with a modulated voice of a first type in which both a pitch and a formant of the voice data are increased, a modulated voice of a second type in which the pitch of the voice data is decreased and the formant of the voice data is increased, a modulated voice of a third type in which the pitch and the formant of the voice data are decreased, and a modulated voice of a fourth type in which the pitch of the voice data is increased and the formant of the voice data is decreased;
receiving any one of the first to fourth types selected by the user;
determining which element of the pitch and the formant the user wants to adjust based on the type selected by the user, which includes providing keywords corresponding to the first to fourth types to the user; and
in response to receiving the keyword selected by the user, adjusting the pitch and the formant of the voice data and providing the adjusted voice data to the user.

17. The psychology counseling method of claim 10, wherein the providing of at least two of the plurality of modulated voices to the user includes further providing the user with the voice that the user articulates the texts.

18. The psychology counseling method of claim 10, wherein the texts provided to the user includes any one of a text based on a positive self-talk (PST), a text based on a cognitive behavioral therapy (CBT) method, and a text based on mindfulness and breathing.
